(19) United States
(12) Patent Application Publication
(10) Pub. No.: US 2025/0161664 A1
(43) Pub. Date: May 22, 2025

(54) SYSTEMS AND METHODS FOR PERIPHERAL NERVE STIMULATION

(71) Applicant: Cala Health, Inc., San Mateo, CA (US)

(72) Inventors: Serena HanYing Wong, Palo Alto, CA (US); Kathryn H. Rosenbluth, San Francisco, CA (US); Samuel Richard Hamner, San Francisco, CA (US); Paula Jean Chidester, Menlo Park, CA (US); Scott Lee Delp, Stanford, CA (US); Terence D. Sanger, Los Angeles, CA (US)

(21) Appl. No.: 19/030,314

(22) Filed: Jan. 17, 2025

Related U.S. Application Data
(63) Continuation of application No. 18/929,457, filed on Oct. 28, 2024.
Application No. 18/929,457 is a continuation of application No. 17/013,396, filed on Sep. 4, 2020, now Pat. No. 12,157,001.
Application No. 17/013,396 is a continuation of application No. 15/580,631, filed on Dec. 7, 2017, now Pat. No. 10,765,856.
(60) Provisional application No. 62/173,894, filed on Jun. 10, 2015.

(51) Int. Cl. A61N 1/04; A61N 1/32; A61N 1/36; A61N 1/372

(52) U.S. Cl. CPC A61N 1/0456; A61N 1/0476; A61N 1/0484; A61N 1/0496; A61N 1/321; A61N 1/36017; A61N 1/36057; A61N 1/36067; A61N 1/36139; A61N 1/36014; A61N 1/36125; A61N 1/37247; A61N 1/37252

(57) ABSTRACT

A system and method for providing therapy to a patient can include a monitoring unit and a therapy unit. The monitoring unit can have a user interface and one or more sensors to measure patient data. The therapy unit can have a stimulator for generating electrical stimulation and a microcontroller for controlling the generation of the electrical stimulation based on the measured patient data.

### CROSS REFERENCE TO RELATED APPLICATIONS

This application is a continuation of U.S. patent application Ser. No. 18/929,457 filed Oct. 28, 2024, which is a continuation of U.S. patent application Ser. No. 17/013,396 filed Sep. 4, 2020 now U.S. Pat. No. 12,157,001, which is a continuation of U.S. patent application Ser. No. 15/580,631 filed Dec. 7, 2017 now U.S. Pat. No. 10,765,856, which is a U.S. National Stage under 37 C.F.R § 371 of PCT App. No. PCT/US2016/037080 filed on Jun. 10, 2016, which claims priority to U.S. Provisional Application No. 62/173,894, filed Jun. 10, 2015, each of which is herein incorporated by reference in their entireties.

### INCORPORATION BY REFERENCE

U.S. Patent Publication No. 2015/0321000, filed Jul. 21, 2015, and International Publication No. WO2015/187712, filed Jun. 2, 2015, and herein incorporated by reference in their entireties for all purposes.

All publications and patent applications mentioned in this specification are herein incorporated by reference to the same extent as if each individual publication or patent application was specifically and individually indicated to be incorporated by reference.

### FIELD

Embodiments of the invention relate generally to systems and methods for treating a disease or disorder, and more specifically to systems and method for treating a disease or disorder, such as tremor, using a monitoring unit and a therapy unit.

### BACKGROUND

Essential tremor (ET) is the most common movement disorder, affecting an estimated 10 million patients in the U.S., with growing numbers due to the aging population. The prevalence increases with age, increasing from 6.3% of the population over 65, to above 20% over 95. ET is characterized by oscillatory movement, for example between 4-12 Hz, affecting distal limbs, especially the hands. Unlike Parkinson's tremor, which exists at rest, essential tremor is postural and kinetic, meaning tremor is induced by holding a limb against gravity or during movement respectively.

Disability with ET is common, and varies from embarrassment to the inability to live independently as key tasks such as writing and self-feeding are not possible due to the uncontrolled movement. Despite the high disability and prevalence of ET, there are insufficient treatment options to address tremor. Drugs used to treat tremor (e.g., Propranolol and Primidone) have been found to be ineffective in 40% of patients and only reduces tremor by 50%. These drugs also have side effects that can be severe. The alternative treatment is surgical implantation of a deep brain stimulator, which can be effective in reducing tremor amplitude by 90%, but is a highly invasive surgical procedure that carries significant risks and cannot be tolerated by many ET patients. There is thus a great need for alternative treatments for ET patients.

Tremor is also a significant problem for patients with orthostatic tremor, multiple sclerosis and Parkinson's disease. The underlying etiology of tremor in these conditions differs from ET, however treatment options for these conditions are also limited and alternative treatment is warranted.

A number of conditions, such as tremors, can be treated through some form of transcutaneous peripheral nerve stimulation. People have a wide variation in wrist diameters, nerve locations, nerve depolarization characteristics, and skin conduction that leads to challenges in designing a device to comfortably, safely and reliably stimulate the peripheral nerves across a broad population of potential users. For instance, in a wrist-worn device targeting the median, ulnar, and radial nerves at the wrist, the amount of power needed for a given stimulation session can vary widely based on skin impedance and usage scenarios.

### SUMMARY OF THE DISCLOSURE

The present invention relates generally to systems and methods for treating a disease or disorder, and more specifically to systems and method for treating a disease or disorder, such as tremor, using a monitoring unit and a therapy unit.

The devices and methods of this disclosure each have several innovative aspects, no single one of which is solely responsible for the desirable attributes disclosed herein. The present application discloses devices and methods for reducing tremor in a subject. In some embodiments, a device is provided. The device can include a housing and one or more affectors, power sources, or controls. In some embodiments, the device further includes one or more sensors. Further aspects and embodiments of the present invention are set forth herein.

These and other aspects and embodiments of the invention are described in greater detail below, with reference to the drawing figures.

In some embodiments, a system for treating tremor of a patient is provided. The system can include a band and a detachable therapy unit. The band can have at least two electrodes, a receptacle, and a first electrical circuit in electrical communication with both the at least two electrodes and the receptacle. The detachable therapy unit can include a second electrical circuit; one or more sensors in electrical communication with the second electrical circuit, the one or more sensors configured to measure data from the patient; a stimulator configured to generate an electrical stimulation, the stimulator in electrical communication with the second electrical circuit; at least two electrodes that are configured to receive the electrical stimulation from the stimulator; a controller configured to control the generation of the electrical stimulation by the stimulator; and a power source in electrical communication with the second electrical circuit; wherein the detachable therapy unit is configured to be reversibly attached to the receptacle of the band such that the at least two electrodes are in electrical communication with the stimulator.

In some embodiments, the band further includes one or more identifiers.

In some embodiments, the one or more identifiers are associated with stimulation parameters and/or usage life information.

In some embodiments, the system further includes a base station configured to charge the power source. In some embodiments, the base station is further configured to receive and transmit data to and from the detachable therapy unit and to and from a cloud computing network.

In some embodiments, the system further includes an online portal, such as a physician web portal, configured to access the data stored on the cloud computing network.

In some embodiments, the system further includes an online portal, such as a physician web portal, configured to provide information and parameter changes back to the detachable therapy unit.

In some embodiments, the system further includes a portable computing device with a second user interface and a display, wherein the portable computing device is configured to wirelessly communicate with the detachable therapy unit and to receive data from the cloud computing network.

In some embodiments, the receptacle comprises a securement feature for reversibly attaching the detachable therapy unit to the receptacle.

In some embodiments, the securement feature is selected from the group consisting of a clip, a magnet, a snap fit mechanism, a twist fit mechanism, a screw mechanism, a latching mechanism, a sliding mechanism, a flexible lip, and a hook.

In some embodiments, the detachable therapy unit further comprises a user interface.

In some embodiments, the controller is configured to control the generation of the electrical stimulation by the stimulator based on data measured by the one or more sensors.

In some embodiments, a system for treating tremor of a patient is provided. The system can include a wearable monitoring unit and a first therapy unit. The wearable monitoring unit can include an electrical circuit; one or more sensors in electrical communication with the electrical circuit, the one or more sensors configured to measure data from the patient; at least two electrodes. The first therapy unit can include a power source; a stimulator powered by the power source, the stimulator configured to generate an electrical stimulation that is delivered through the at least two electrodes of the wearable monitoring unit; and a controller configured to control the generation of the electrical stimulation by the stimulator based on data measured by the one or more sensors; wherein the first therapy unit is reversibly attachable to the wearable monitoring unit.

In some embodiments, the system further includes a second therapy unit. The second therapy unit can include a second power source, wherein the second power source of the second therapy unit has more electrical capacity than the power source of the first therapy unit; a second stimulator powered by the second power source, the second stimulator configured to generate an electrical stimulation that is delivered through the at least two electrodes of the wearable monitoring unit; and a second controller configured to control the generation of the electrical stimulation by the stimulator based on data measured by the one or more sensors; wherein the second therapy unit is reversibly attachable to the wearable monitoring unit.

In some embodiments, the one or more sensors are configured to measure motion data. In some embodiments, the controller is configured to determine the tremor frequency, amplitude, and/or phase from the motion data; and control the generation of the electrical stimulation by the stimulator based on the determined tremor frequency, amplitude, and/or phase.

In some embodiments, the at least two electrodes are disposed on a band. In some embodiments, at least one of the at least two electrodes is disposed on a band that is attached to a housing of the wearable monitoring unit and at least one of the at least two electrodes is disposed on a skin facing side of the housing of the wearable monitoring unit.

In some embodiments, a system for treating tremor of a patient is provided. The system can include a wearable monitoring unit and a therapy unit. The wearable monitoring unit can include a user interface; an electrical circuit in electrical communication with the user interface; and one or more sensors in electrical communication with the electrical circuit, the one or more sensors configured to measure data from the patient. The therapy unit can include a stimulator configured to generate an electrical stimulation; at least two electrodes that are configured to receive the electrical stimulation from the stimulator; a controller configured to control the generation of the electrical stimulation by the stimulator based on data measured by the one or more sensors; and a power source disposed within the wearable monitoring unit or the therapy unit; wherein the therapy unit is reversibly attachable to the wearable monitoring unit.

In some embodiments, the wearable monitoring unit is a smart watch.

In some embodiments, the one or more sensors are configured to measure motion data. In some embodiments, the controller is configured to determine the tremor frequency, amplitude, and/or phase from the motion data; and control the generation of the electrical stimulation based on the determined tremor frequency, amplitude, and/or phase.

In some embodiments, the controller is configured to provide automatic and/or manual control of the electrical stimulation.

In some embodiments, the wearable monitoring unit further includes a controller configured to determine the tremor frequency, amplitude, and/or phase from the motion data, and the controller of the therapy unit is configured to control the generation of the electrical stimulation by the stimulator based on the determined tremor frequency, amplitude, and/or phase.

In some embodiments, the at least two electrodes are disposed on a band. In some embodiments, at least one of the at least two electrodes is disposed on a band that is attached to a housing of the therapy unit and at least one of the at least two electrodes is disposed on a skin facing side of the housing of the therapy unit.

In some embodiments, the therapy unit communicates wirelessly with the wearable monitoring unit.

In some embodiments, both the therapy unit and the wearable monitoring unit each have a power source.

In some embodiments, the at least two electrodes are covered with a porous, compressible material that is impregnated with a conductive gel, wherein the porous, compressible material is configured to release the conductive gel when pressure is applied to the porous, compressible material.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate various embodiments of a monitoring unit and a therapy unit that form a two part treatment system.

FIGS. 2A-2D illustrate an embodiment of a two part system with a single monitoring unit and a plurality of therapy units.

FIG. 3 illustrates an embodiment of a two part stimulation where both the monitoring unit and the therapy unit are bands.

FIGS. 4A and 4B illustrate an embodiment of a touch sensor array that can be used to select electrodes from a corresponding electrode array.

FIG. 5 illustrates an embodiment of a two part system formed from a monitoring patch and therapy unit.

FIGS. 6-8 illustrate various embodiments of skin interfaces that incorporate a conductive gel.

FIGS. 9A-9I illustrate another embodiment of a wearable therapy system.

FIG. 10 illustrates an embodiment of the wearable therapy system that uses the cloud to receive and transmit data between the therapy system and a physician.

FIG. 11 is a block diagram that illustrates the individual components of the therapy unit, band, and base station shown in FIG. 10.

### DETAILED DESCRIPTION

Embodiments of the invention include a device and system and method to measure and collect motion and biological data (e.g., heart rate, galvanic skin response, temperature, and characteristics of the motion disorder, such as tremor frequency, amplitude, and phase), analyze the data as to interpret how these measures may influence motion disorders such as tremor or freezing of gait, and provide peripheral nerve stimulation that targets one or more individual nerves to reduce tremor or initiate gait, where the stimulation applied may or may not be modified based on the measured data.

Embodiments of the therapy system can include three components: (1) a monitoring unit having sensors, circuitry, and optionally may have a power source and/or a microcontroller, (2) a therapy unit having a stimulator (e.g., a pulse generator), circuitry, a power source and a microcontroller, and (3) a skin interface having electrodes and electrical connections for electrically connecting the electrodes to the therapy unit. In some embodiments, all three components are separate components that can be reversibly attached to each other to form a wearable therapy system. In some embodiments, any two of the components can be combined or integrated together to form a wearable two part system that can be reversibly attached to each other. It should be noted that some functions can crossover, such as the electrodes of the skin interface being used as sensors to measure electrical activity (e.g. EMG and ECG) and impedance, for example. In some embodiments, any one of the detachable components can be disposable and/or can be sent back to the manufacturer for recycling.

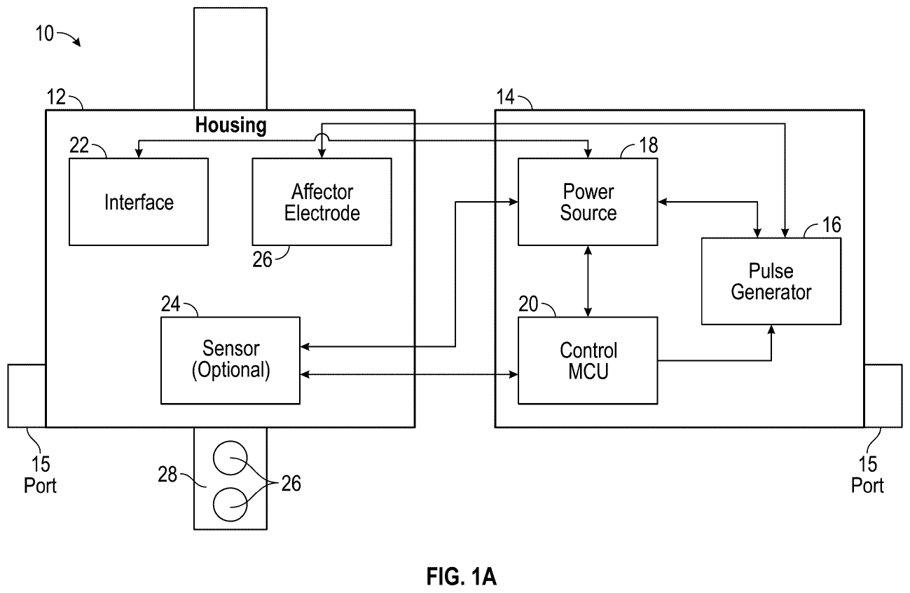

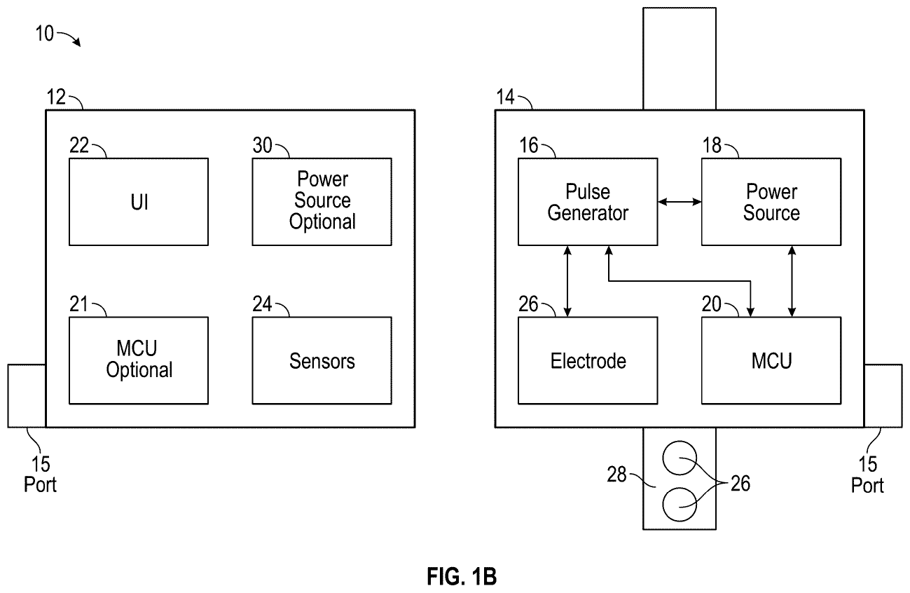

One embodiment, as shown in FIG. 1A, is a two-part system 10 including a I) a monitor unit 12 that can be wearable in some embodiments and 2) a therapy unit 14. In some embodiments, the therapy unit 14 can be detachable and can be reversibly attached to the wearable monitor unit 12. The therapy unit 14 may contain an electrical stimulation signal generator 16, power source 18, and a microprocessor and/or microcontroller 20 to control the stimulation. The therapy unit 14 can reversibly connect and communicate directly and/or wirelessly to the wearable monitor 12. In some embodiments, the therapy unit 14 may remain separate from the wearable monitor unit 12 and can communicate wirelessly with the wearable monitor unit 12. In some embodiments, the therapy unit 14 can have a data/power port 15, such as a USB port that allows a user to charge the power source 18, update the software and/or parameters on the microcontroller 20, and/or retrieve data from memory on the wearable monitor unit 12 and/or therapy unit 14. In some embodiments, the data/power port can be located on the wearable monitor unit 12 or both the wearable monitor unit 12 and therapy unit 14. In some embodiments, the wearable monitor unit 12 and/or therapy unit 14 can communicate wirelessly with an external computing device to update the software and/or parameters and/or retrieve data.

In some embodiments, the wearable monitor unit 12 can have a housing with a user interface 22 that encloses one or more sensors 24. In some embodiments, the wearable monitor 12 can be used to detect and/or measure tremor. In some embodiments, the wearable monitor 12 can have one or more electrodes 26 located on the base of the housing that makes contact with the patient's skin. In addition or alternatively, the wearable monitor 12 can have a band 28 or other securement feature with one or more electrodes on the skin facing side of the band 28. In some embodiments, the wearable monitor unit 12 has 2 or 3 electrodes, or at least 2 or 3 electrodes. In some embodiments, the wearable monitor unit 12 lacks a power source and relies on the power source 18 in the therapy unit 14 for power. In other embodiments, both the wearable monitor unit 12 and the therapy unit 14 have power sources. In some embodiments, only the wearable monitor unit 12 has a power source and the therapy unit relies on power from the monitoring unit.

In some embodiments, as shown in FIG. 1B, the therapy unit 14′ may directly make contact with the wearer's skin and have the capability to provide electrical stimulation of targeted nerves, such as the median and radial nerves or the tibial nerve or the sacral nerve, using electrodes 26. In some embodiments, the therapy unit 14′ has 2 or 3 electrodes, or at least 2 or 3 electrodes. These electrodes 26 may be located on the housing of the therapy unit 14′ and/or the therapy unit 14′ may also have a band 28 or securement feature with electrodes 26. In some embodiments, when the therapy unit 14′ has electrodes 26, the wearable monitor unit 12′ does not have electrodes. In some embodiments, both the monitor unit and the therapy unit can have electrodes. As above, the therapy unit 14′ can have a stimulator 16, power source 18, and microcontroller 20. The wearable monitor unit 12′ can have a user interface 22 and one or more sensors 24 and, optionally, a power source 30 and microcontroller 21. In some embodiments, when the monitor unit has a power source 30 and/or a microcontroller 21, the therapy unit does not have a power source and/or a microcontroller. In some embodiments, the wearable monitor unit 12′ is a smart watch, such as the Apple Watch or an Android based smart watch, with an application that allows the smart watch to communicate with the therapy unit and perform as a monitor unit. In some embodiments, the wearable monitor unit 12′ can communicate with the therapy unit 14′ wirelessly, and one or both of these devices can also communicate with an external computing device wirelessly. In some embodiments, one or both of the wearable monitor unit 12′ and the therapy unit 14′ can have a data/power port 15. In some embodiments, the wearable monitor unit 12 and the therapy unit 14′ can be connected to each other through the data/power ports 15.

In some embodiments, the sensors can be located in or on the therapy unit instead of the monitoring unit. In some embodiments, the sensors can be located on both the therapy unit and the monitoring unit.

In some embodiments, the monitor unit can instead be carried by the user in, for example, the user's hand or pocket, rather than be worn. For example, a monitor unit carried by the user can be a smart phone, such as an Android smartphone or iPhone.

In some embodiments, the two part system or the monitor unit may instruct the user to perform an action, such as draw, write, or hold an object, or to remain still or to attempt to remain still while the wearable monitor unit takes a measurement with one of the sensors.

In some embodiments, the user interface can include a display. In some embodiments, the display can be a touch screen display. In some embodiments, the user interface can include one or more buttons and/or a keyboard.

In some embodiments, the electrodes can be dry-contact (e.g., fabric, metal, silicone or any other plastic impregnated with conductive fillers, or a combination), use a conductive gel (e.g., hydrogels), or have a wet electrode surface (e.g., a sponge with water or conductive liquids or gels), or have fine micro needles, for example. In some embodiments, the electrodes can have a foam backing as further described below.

In one embodiment of the system, the stimulation is provided by implanted electrodes that stimulate nerves in the wrist, such as the median nerve or radial nerve, or other nerves in a plurality of other locations, such as nerves in the leg like the tibial nerve, or nerves in the back like the sacral nerve. The implantable electrode may be powered by a rechargeable battery housed within the implant and recharged wirelessly from an external power source.

In another embodiment of an implanted electrode that stimulates the nerve, the implanted electrode is powered by an external therapy unit, and the stimulation pulse is directly coupled to the electrode and nerve using capacitive or inductive coupling.

In some embodiments, the monitor unit can be a wearable tremor monitor having a housing with a user interface. The housing use a plurality of sensors to collect, store, and analyze biological measures about the wearer including, but not limited to, motion (e.g., accelerometers, gyroscopes, magnetometer, bend sensors), muscle activity (e.g., EMG using electrodes), cardiovascular measures (e.g., heart rate, heart rate variability using electrodes to measure ECG, heart rhythm abnormalities), skin conductance (e.g., skin conductance response, galvanic skin response, using electrodes), respiratory rate, skin temperature, and sleep state (e.g., awake, light sleep, deep sleep, REM). In particular, studies have shown that increased stress levels can increase tremor in people with Essential Tremor, Parkinson's Disease, and other diseases causing tremor. Thus, using standard statistical analysis techniques, such as a logistical regression or Naive Bayes classifier, these biological measures can be analyzed to assess a person's state, such as level of stress, which in turn, can serve as a predictor for increases in tremor level. In an early pilot study, patients were asked to perform activities prior to and after a stressful event. In this case, the stressful event was to take a timed math test. In preliminary studies, the patients' amplitude of tremor appeared to increase by about 20% after the stressful timed math test.

In one embodiment of the wearable monitor, the skin interface has an array of microneedles. Microneedles have been shown to measure blood chemistry using electrochemical sensors that can be used to detect specific molecules or PH levels. Thus the monitoring unit could incorporate microneedles with electrochemical sensors to measure specific chemicals in the blood stream that may affect tremor, such as stress hormones, caffeine, or medications.

In one embodiment of the monitor, a saliva sample is taken with a paper strip placed in the mouth, and saliva chemistry is analyzed by sensors in the wearable monitor or in a standalone analysis unit, for substances that may affect tremor, including stress hormones (e.g., cortisol), caffeine, or medications. The unit could have a light source and photo detectors to analyze the chemistry of the strip. The unit could also communicate with an external processing device, such as a cell phone. The strips could be visually coded to record and store information about the measurement (e.g., time, location, etc.).

The wearable tremor monitor can have a microprocessor to analyze biological measures about the wearer to: determine or predict the onset of increased tremor activity, set parameters of the stimulation waveform applied by the therapy unit, and/or adapt the stimulation waveform applied by the therapy unit in real time. Parameters of the stimulation waveform that could be modified based on analysis of biological measures are frequency, amplitude, shape, burst sequence. In some embodiments, the analysis can be performed by a microprocessor on the therapy unit or an external computing device.

One embodiment of the system could centrally store biological measures from multiple wearers on a server system (e.g., the cloud), along with other relevant demographic data about each user, include age, weight, height, gender, ethnicity, etc. Data collected from multiple wearers is analyzed using standard statistical techniques, such as a logistic regression or Naive Bayes classifier (or other classifiers), to improve prediction of tremor onset by determining correlations between biological measures and other recorded events and onset of increased tremor activity. These correlations are used to set parameters of the stimulation waveform applied by the therapy unit, determine best time to apply stimulation therapy, and/or adapt the stimulation waveform applied by the therapy unit in real time.

In one embodiment of the system, the wearable tremor monitor that automatically detects and records the dosage and consumption of medications to (1) track compliance of the patient; (2) combine with the measurement of tremor activity to assess therapeutic effectiveness, and (3) determine or predict the onset of an increase or decrease in tremor activity. The dosage and consumption of medications can be detected and record in multiple ways, including (1) using visual scanner to record a marking on the pill pack or bottle each time medication is consumed, (2) a smart pill cap with force sensors and a wireless transmitter to detect each time the medication is consumed from a pill bottle, (3) an RFID chip that is of similar size and shape as a pill that is consumed with each dosage of medication that is activated by digestion and communicates with the monitor device, (4) an RFID chip embedded in a sugar pill that is consumed with each dosage of medication that is activated by digestion and communicates with the monitor device, and (5) a pill with a visual encoding that is scanned and recorded by a camera on the monitor unit each time medication is consumed.

In some embodiments, the wearable tremor monitor can have a visual, auditory, tactile (e.g., squeezing band), or vibrotactile cues to notify the wearer of key events based on analysis of biological measures, including, but not limited to, prediction of tremor onset, increase in tremor activity, and/or increase in stress level. The cuing system could also notify the wearer of other predetermined events or reminders set by the wearer. Cuing system is used to (1) communicate information to the wearer, such as onset of increased tremor activity or other predetermined events, in a more discreet, personalized way, without drawing attention from others in social situations.

In some embodiments, the form of the wearable monitor and/or therapy unit could be a wrist band or watch, a ring, a glove, an arm sleeve or arm band, an ear piece/headphone, head band, a necklace or neck band, or a compliant patch.

In one embodiment, the wearable monitor can have a processing unit and memory that collects, stores, processes, and analyzes the biological measures, along with other data input by the wearer.

In some embodiments, the wearable monitor can take user input about events, including diet history, medication history, caffeine intake, alcohol intake, etc. The monitor can use accelerometers to measure specific movements, gestures, or tapping patterns to record user inputs at specific prompts. Other touch sensors, such as resistive strips or pressure sensitive screens, could be used to measure specific gestures to record user inputs. These gesture based measures to record user input minimize the complexity of steps required to input user data into the device. The data can be stored in memory and processed by the processing unit. In some embodiments, the data can be transmitted from the wearable monitor to an external computing device.

In one embodiment, the wearable monitor and/or the therapy unit can connect with other applications, such as calendars and activity logs, to sync and track events or a saved calendar can be saved and stored on the device. In some embodiments, the wearable monitor and/or the therapy unit can communicate with a variety of computing devices, such as a smart phone, a smart watch, a tablet, a laptop computer, or a desktop computer, for example, that have these applications.

In one embodiment, the monitor unit and/or therapy unit can have a GPS or similar device to track the location and assess activity of the wearer. GPS measures can be combined with mapping or location systems to determine context of the wearer's activity (e.g., gym, office, home) or determine changes in elevation during specific activities, such as running or cycling.

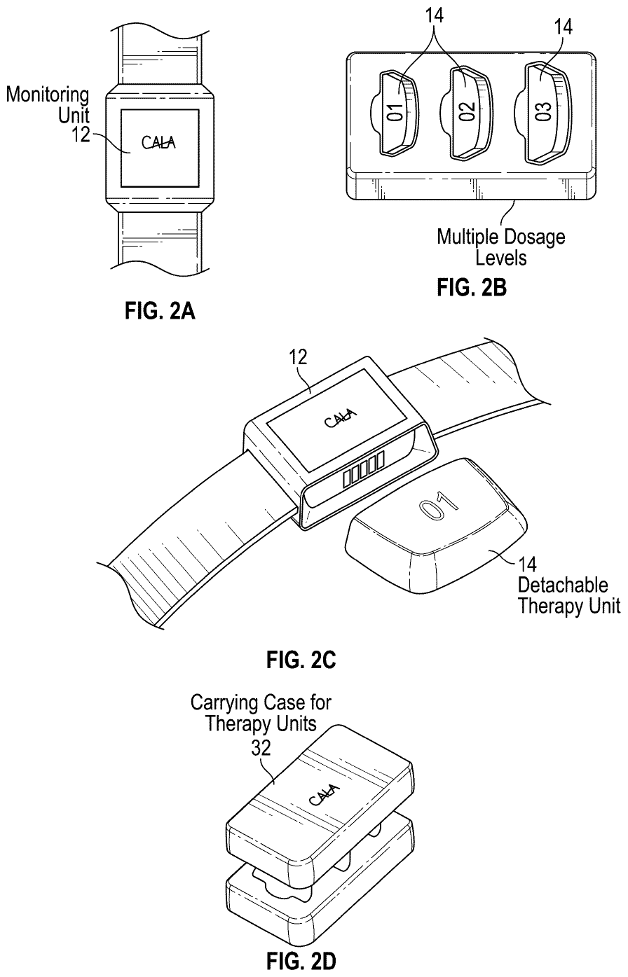

In some embodiments as shown in FIGS. 2A-2D, a single monitor unit 12 can be used with a plurality of therapy units 14 having different sizes, shapes, colors, markings and/or capabilities, which includes different battery capacity and power output. Different wearers and usage scenarios may require different amounts of stimulation duration and power, making a smaller or larger therapy unit more desirable and giving the wearer options to meet their needs in different scenarios. In some embodiments, the therapy units 12 can also have different programming, including different stimulation parameters and/or therapies which can be tailored to different types of treatments. For example, one therapy unit can be tailored to treat essential tremor, while another therapy unit can be used to treat Parkinson's disease and another for freezing of gait or overactive bladder. In some embodiments, the therapy units can each be tailored to provide different intensity of treatments, such as one unit for light treatment of essential tremor and another for heavy and aggressive treatment of essential tremor. The different features and capabilities of the therapy units can correspond to the different sizes, shapes, color, and/or markings. A carrying case 32 can be used to hold a set of therapy units, such as a set of therapy units to treat essential tremor that differ in battery capacity and power output or some other feature.

In one embodiment, the therapy units have a unique charging station that can simultaneously charge multiple therapy units. The charging station could have a custom direct electrical connection to the therapy units or could charge the therapy units wirelessly in a close proximity. Similarly, in some embodiments, the charging station can charge the monitoring units in a similar manner.

In one embodiment, the wearable monitor can track parameters about stimulation provided by the therapy unit, including time of stimulation, duration of the stimulation session, and power used by the therapy unit. This data can be stored on memory in the wearable monitor, processed by the wearable monitor, and/or transmitted to an external computing device.

In one embodiment, the therapy unit can use switches or an electrical sensor to detect connection of electrodes: (1) to ensure proper and unique electrodes are being installed (i.e., not using a different or incorrect type of electrode) communicating a unique code, for example via RFID, an encoded EEPROM chip, a resistance or capacitance based ID, a binary identifier, or a surface pattern (2) to regulate the number of uses for each electrode to prevent over use, and (3) to prevent the usage of the device without an electrode to prevent small shock. In some embodiments, the therapy unit and/or the monitor unit can have an identifier that can be transmitted to and be received by each other or to an external computing device. The identifier can allow one unit to determine the features, capabilities, and/or configuration of the other device, including the electrode configuration described above, so that the appropriate treatment parameters can be used, and also the usage life or expiration of the component, which can be based on voltage measurements, time, number of therapy sessions, or other parameters. In some embodiments, instead of using an identifier, the features, capabilities, and/or configuration of one device can be transmitted to the other device, either directly from one device to the other device, or through entry into the user interface, or through an external computing device.

Other components of the therapy system, including the band, the therapy unit, the monitoring unit, the skin interface, can each have one or more identifiers that performs the functions described above. These identifiers can encode a variety of information as described herein, as well as predetermined dosing regimens, initialization routines, calibration routines, or specific parameters. The identifiers may be associated with a lookup table that stores the encoded information.

In some embodiments, the wearable monitor and/or the therapy unit can communicate with an external computer or device (e.g., tablet, smartphone, smartwatch, or custom base station) to store data. Communication between the monitor and external device can be a direct, physical connection, or with a wireless communication connection such as Bluetooth or GSM or cellular.

In one embodiment of the device, the therapy unit has an array of electrodes and one or more sensors, such as pressure sensors, between the therapy unit and the wearer's wrist to measure pressure of contact of the skin interface at and/or around the electrodes. This pressure data can be analyzed to determine which electrodes in the array to stimulate to target the appropriate nerves or to detect changes in skin contact due to motion or other conditions and switch stimulation of the electrode array to the optimal location. These methods are used to (I) assess poor contact of electrodes, and (2) adjust amplitude of stimulation based on pressure measurement.

Increasing contact pressure between the device and the wearer's skin and/or stimulating with electrodes with an adequate contact pressure or above a contact pressure threshold could: (1) increase the surface area of contact, which reduces discomfort, (2) activate deep somatic pain (i.e., type C) peripheral nerve fibers, which could reduce discomfort from stimulation, which activates superficial pain (i.e., type A delta) fibers, (3) reduce the stimulation amplitude needed because it improves stimulation of the targeted nerve (e.g., the electrode is physically closer to the nerve by pressing it), or (4) reduce the effect of skin motion.

In one embodiment, the therapy unit has the form of an inflatable wrist band, which is made of a pliable, airtight material. A small pump is actuated or activated by the user to fill the bladder with air and increase pressure to increase the surface area of contact, which reduces discomfort. In some embodiments, the pump is integrated into the wrist band and can be either mechanically actuated by the user or electrically powered by a battery. In other embodiments, the pump can be separate from the wrist band.

In one embodiment, the pressure is provided by a compliant material within the band, such a soft open cell foam or an array of mini springs (e.g., pogo pins).

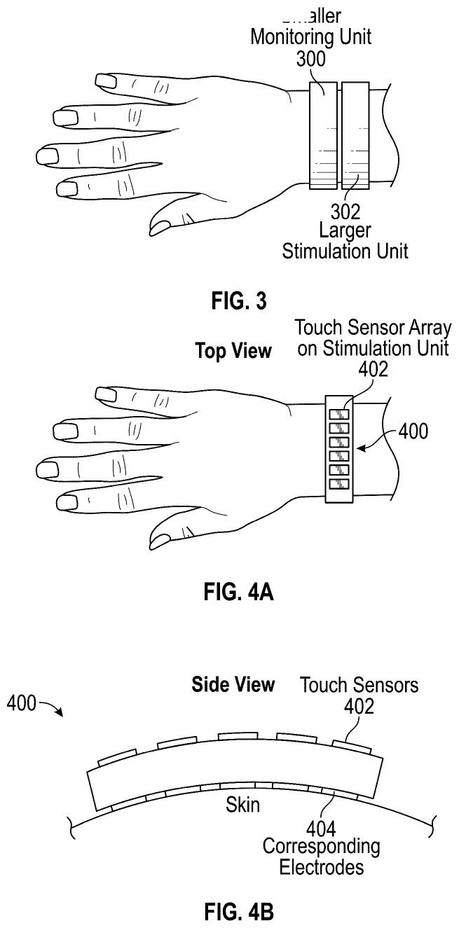

In one embodiment of the device as shown in FIG. 3, the monitor unit 300 and the therapy unit 302 have the form factor of two distinct wrist bands that can connect to each other. The monitor unit 300 is the primary wrist band, and the therapy unit 302 is attached secondarily, as needed, into the monitor unit 300. Alternatively, the therapy unit 302 may remain separate of the monitor unit 300 and can communicate wirelessly with each other.

In one embodiment as shown in FIG. 4, the therapy unit 400 has a touch sensor array 402 that corresponds to an electrode array 404. When attached to the wearer, the electrode array 404 is contacting the wearer's skin, and the touch sensor array 402 is on the opposite, outer part of the therapy unit 400. The wearer can use the touch sensor to indicate the preferred location of stimulation of the therapy unit 400 by touching the desired location on the touch sensor. For example, touching one touch sensor in the touch sensor array 402 activates the corresponding electrode in the electrode array 404, allowing the user to easily select which electrodes of the electrode array 404 to use for stimulation.

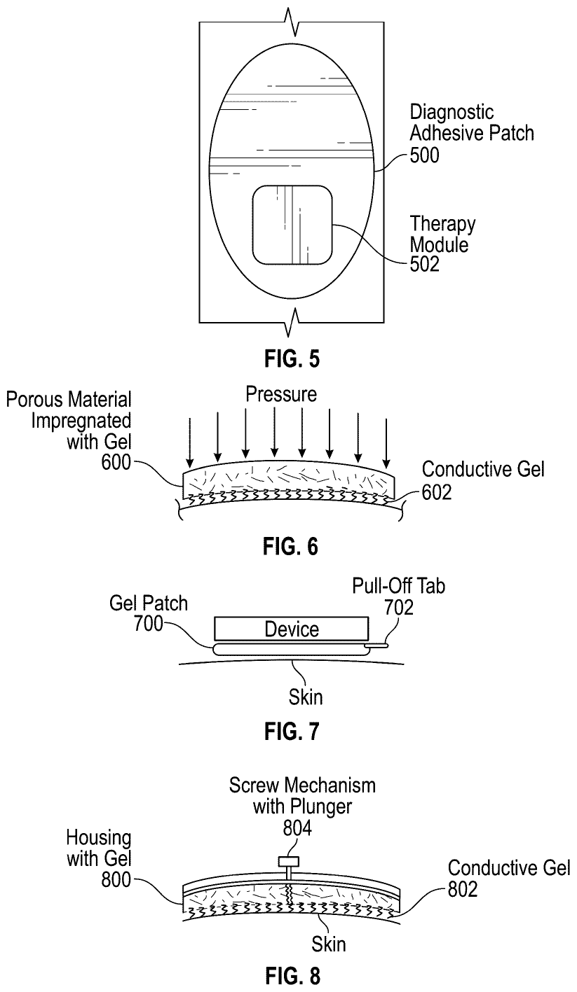

In some embodiments as shown in FIG. 5, the two part system can include a monitor unit that is a wearable monitor patch 500 with at least two electrodes or at least two separate patches each with an electrode that adheres to the skin via an adhesive, surface adhesion of the material, or microneedles. The monitor patch also can include a current spreader at the skin interface to deliver electrical energy to the skin more evenly. Generally, it is desirable to have at least two electrodes or patches in order to have a ground electrode to adequately deliver energy to nerves transcutaneously. The system can also have a therapy unit 502 that houses the power source, signal generator, and microcontroller for electrical stimulation through the monitor patch 500 to which it attaches via a metal snap, a magnet or other electrical connector.

In one embodiment of the above, the therapy unit wirelessly powers the monitoring patch.

In some embodiments as shown in FIG. 6, the skin interface at and/or around the electrode of the monitoring unit and/or therapy unit is provided by a porous material 600, such as a foam polymer, that is impregnated with conductive gel 602. In some embodiments, the foam polymer is made of a conductive polymer. In other embodiments, the foam polymer is made of a nonconductive polymer. In some embodiments, the foam material is flexible and compressible and can conform to the patient's skin. The conductive gel is disposed within the pores of the porous material, and as pressure is applied to the porous layer by for example the housing and electrode, conductive gel is pushed and squeezed out through the pores of the porous material and onto the wearer's skin as the foam material is compressed. Pressure can be applied to the porous material by squeezing and/or pushing on the housing of the therapy unit against the skin by the wearer, or by a screw mechanism, or some other mechanism that tightens the outer housing to compress the gel layer, or by tightening a band that fastens the therapy unit to the patient.

In some embodiments as shown in FIG. 7, the skin interface of the housing and electrodes of the therapy unit and/or monitoring unit can include a thin gel patch 700 covered by a protective liner with a pull-off tab 702 that slides between and/or is positioned between the device and the wearer's skin. The protective liner can be removed using the pull-off tab 702 to expose the conductive gel. The gel patch is electrically conductive and creates and/or improves an electrical connection between the skin and the electrodes to deliver electrical energy to the wearer's skin.

In some embodiments as shown in FIG. 8, the housing of the therapy unit and/or monitoring unit and/or structure with the electrodes, such as a band, can include a reservoir 800 filled with a conductive gel 802 and a plurality of pores or channels at the skin interface through which the conductive gel in the reservoir can be dispensed onto the wearer's skin to improve the skin interface for stimulation. The gel can be dispensed with a screw mechanism 804 or plunger or button or collapsible reservoir or other mechanism that pushes gel out of the reservoir through small channels or pores onto the wearer's skin.

In one embodiment, the system helps the wearer relax—by using a cuing system to remind the wearer to relax or practice relaxation techniques. The cuing can be auditory, visual, or tactile. Also, the system can provide feedback about the wearer's stress level that gives reinforcement that relaxation techniques are working.

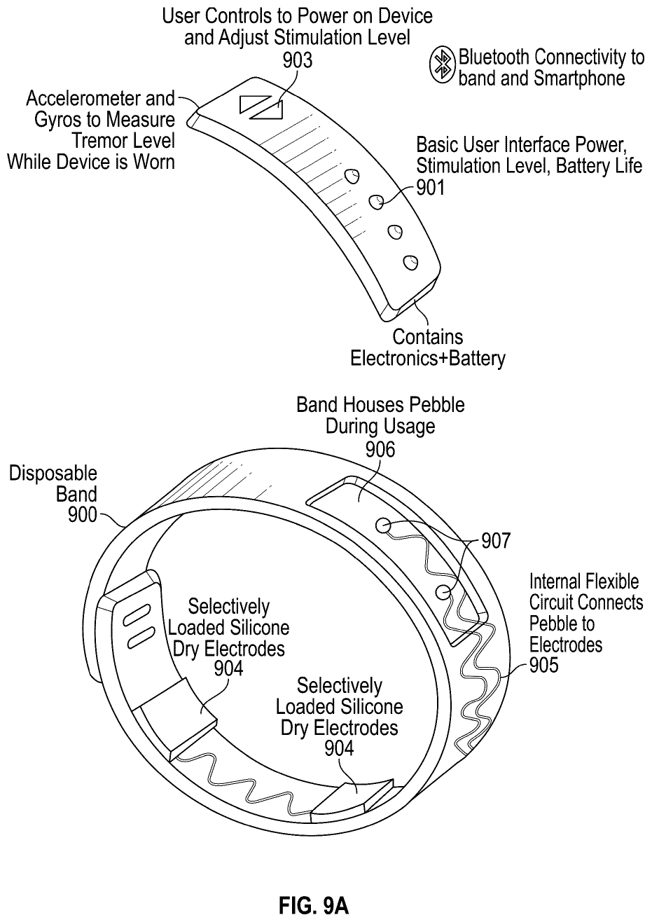

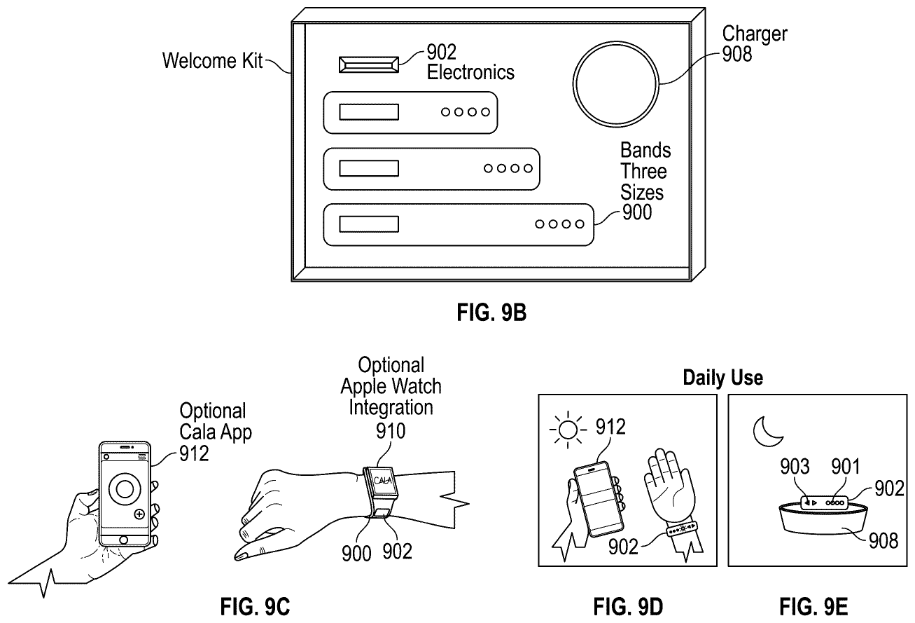

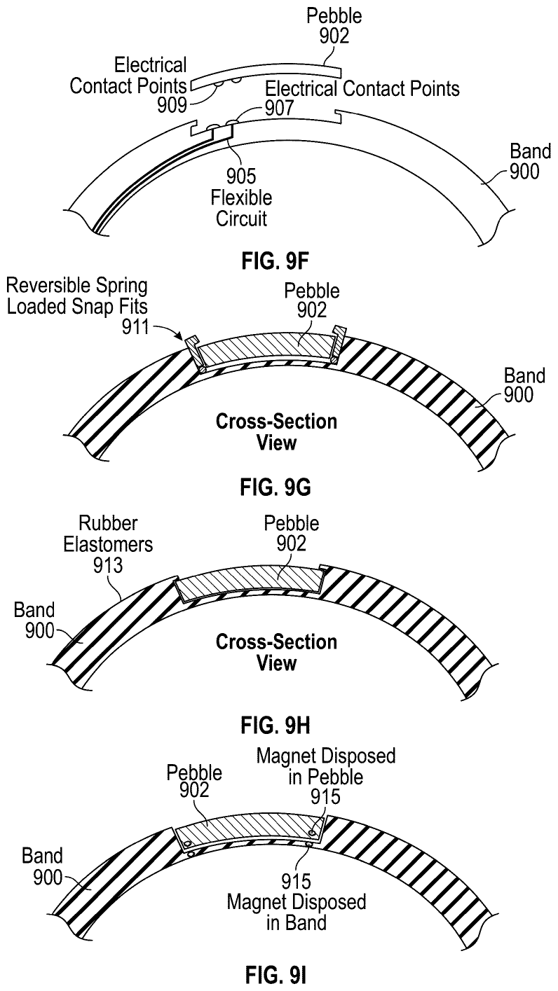

FIGS. 9A-9I illustrates another embodiment of a two part therapy system that includes a disposable band 900 and a therapy unit 902 that can be reversibly attached to the disposable band 900. The disposable band 900 can have two or more electrodes 904 disposed on a skin facing or inside surface of the band and a receptacle 906 or receiving portion for reversibly receiving the therapy unit 902. Within the band 900 are wires and/or conductive traces that form a flexible circuit 905 that runs from the electrodes 904 to the receptacle 906 for electrically connecting the electrodes 904 to the therapy unit 902 when the therapy unit 902 is disposed in the receptacle 906. In some embodiments, the wires and/or conductive traces of the flexible circuit 905 are arranged in a wave or undulating pattern in order to improve its ability to flex. In some embodiments as shown in FIG. 9F, the receptacle 906 can have one or more electrical contact points, such as one or more pin holes 907, for receiving one or more complementary electrical contacts, such as pins 909, from the therapy unit 902. The flexible circuit 905 can extend to the pin holes 907 such that an electrical connection is formed when the pins are inserted into the pin holes. In some embodiments, as shown in FIGS. 9G-9I, the receptacle 906 can have a clip, retaining lip, magnet, a snap fit, a twist fit, a hook, a latch, a sliding mechanism, or other securement feature for reversibly securing the therapy unit 902 to the band 900. FIG. 9G illustrates clips 911 that may or may not be spring loaded to form a snap fit around the therapy unit 902. FIG. 9H illustrates a flexible lip 913 around the opening of the receptacle that can be used to retain the therapy unit 902 after it is inserted into the receptacle 906. FIG. 91 illustrates magnets 915 that can be placed in complementary positions in the therapy unit 902 and the receptacle. In some embodiments, the clip, magnet, snap fit mechanism, twist fit mechanism, hook, or other securement feature is made of metal or some other conductive material and can be electrically connected to the electrodes via the wires and/or conductive traces. The electrodes 904 can be dry electrodes or can be coated with a conductive gel.

In some embodiments, the therapy unit 902 can include a battery, which may be rechargeable, and electronics to deliver electrical stimulation through the electrodes to the patient's nerves. The electronics can include a stimulator and a microcontroller, and may also include memory and one or more sensors, such as one or more accelerometers and gyroscopes as described herein. In some embodiments, the device is able to sense the impedance of the electrodes in order to assess the integrity of the electrode to skin interface. In some embodiments, there can be an electrical indication (e.g. reading of a chip, pushing in of a sensor on the connector, etc.) to detect integrity of the connection between the band and the therapy unit. In some embodiments, the therapy unit 902 can have one or more LEDs, mini OLED screens, LCS, or indicators 90 I that can indicate the status of the therapy unit 902, such as whether the therapy unit 902 is connected to the band 900, the power remaining in the battery of the therapy unit 902, whether a stimulation is being delivered, the stimulation level, whether data is being transmitted, whether a sensor measurement is being taken, whether a calibration routine is being performed, whether the therapy unit 902 is initializing, whether the therapy unit 902 is paired with another device such as a smart watch and/or smart phone, whether the battery is being charged, and the like. In some embodiments, the therapy unit 902 may also include a user interface 903, such as one or more buttons.

FIG. 9B illustrates a kit that can be sent to a user. The kit can contain a plurality of bands 900 of different sizes, shapes, colors, etc. to accommodate patients having different wrist sizes or other body part sizes, such as ankles, arms, fingers, and legs and to accommodate different types of connected accessories like secondary displays (e.g. smart watch, iwatch). In some embodiments, the kit has three bands. Additionally, the kit can contain one or more electronic units 902. If multiple electronic units 902 are provided in the kit, the battery capacity of the different electronic units 902 can be different to accommodate different usage types. For example, a relatively low capacity battery can be used for on-demand stimulation, while a relatively high capacity battery can be used for automated and/or responsive stimulation driven by the microcontroller. In some embodiments, only a single electronic unit is provided. In other embodiments, a plurality of electronic units are provided while a single band is provided. The kit may also include a charger 908 to charge the therapy unit 902. In some embodiments, the charger 908 can inductively charge the therapy unit 902. In other embodiments, the charger 908 can charge the therapy unit with a charge cable that can be inserted into a power port in the therapy unit. In some embodiments, the therapy unit 902 can be docked with the charger 908 for charging.

FIG. 9C illustrates an embodiment where a smart watch 910, such as the Apple Watch, is reversibly or permanently fastened to a band 900, which may also have a therapy unit 902. In some embodiments, the smart watch 910 may provide a display and a user interface for the therapy unit 902. The smart watch 910 may communicate with the therapy unit 902 wirelessly, such as through Bluetooth or WiFi, or through a direct connection through a data port in the smart watch and a data port in the therapy unit 902. In some embodiments, the electronic unit 902 and/or smart watch 910 may communicate with a smart phone 912, as described herein, to transmit data or to update the software and/or stimulation parameters on the therapy unit 902 and/or smart watch 910. In some embodiments, the band 900 and therapy unit 902 are permanently affixed or integrated together while the smart watch 910 is reversibly attachable to the band 900. The smart phone 912 and/or the smart watch 910 can include an application, which may be downloaded through the cloud or a computer, configured to interface with the therapy unit 902.

FIGS. 9D and 9E illustrate that the wearable two part system can be worn and used throughout the day. When the power remaining in the battery of the therapy unit is low, the therapy unit 902 can be recharged with the charger 908. Charging can be performed at night or whenever the battery is low or when desired. In some embodiments, the therapy unit can be removed from the band before charging. In some embodiments, the user can swap a low charge therapy unit with a high charged therapy unit so that the user can always be wearing a therapy unit.

In some embodiments, the kit illustrated in FIG. 9B can be used as a diagnostic trial kit. The patient can initially wear the therapy system for about 1 to 14 days, or about 1 week, or for a predetermined length of time, with the therapy turned off so that no electrical stimulation is provided to the patient during this time. This initial period is used to collect data with the sensors in the therapy unit and/or band in order to characterize the tremor or other disease. The sensor data can be stored in memory in the therapy unit, and/or can be transmitted through a network to the cloud or a server or to another computing device, which can be accessed by the patient's physician.

Following the data collection phase, the patient can turn on the therapy function on the therapy unit and perform patient-directed tasks after and/or while being given one or more therapy treatments, which may be stored on the therapy unit, in order to identify how well the patient is responding to the various treatments. The patient response data can also be stored on memory and/or transmitted through a network or to another computing device, which can be accessed by the patient's physician.

In some embodiments, the patient can return the kit to the physician or manufacturer of the kit, and data can be retrieved from the system and transmitted to the patient's physician.

Using the data from system, the physician can characterize the patient's tremor or other disease, generate a diagnosis, and determine the appropriate treatment for the patient, which may include selection of the appropriate therapy system and stimulation parameters.

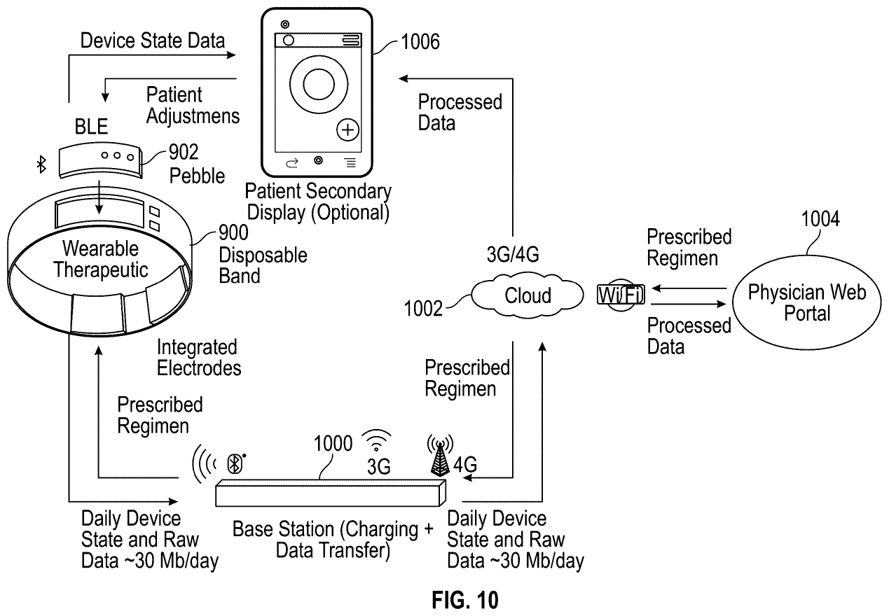

FIG. 10 illustrates an embodiment of a system for treating tremor or another disease or condition using a wearable therapy device. As described above, the therapy device may have two parts, a band 900 and therapy unit 902. A base station 1000, which may replace the charger in the kit described above, can be used to both charge the therapy device and to receive and transmit data to the therapy device and to the cloud 1002. Communication between the base station 1000 and the therapy device can be wireless, such as through Bluetooth and/or WiFi, and communication between the base station 1000 and the cloud 1002 can be through a cellular network, using a 3G or 4G connection, or through a wired connection to the internet, using DSL or cable or ethernet, for example. A physician or other user can view and/or retrieve data stored on the cloud 1002 using an online portal or a physician web portal 1004. In addition, the physician can prescribe and/or modify a treatment regimen on the therapy unit 902 through the cloud 1002 and base station 1000 using the web portal 1004.

In some embodiments, the base station 1000 is used to receive and transmit relatively large amounts of data that may require a high bandwidth, such as the transmission of raw data from the therapy device, which may be about 10 to 100 Mb/day, or about 10, 20, 30, 40, or 50 Mb/day. In some embodiments, the data may be stored in memory in the base station 1000 and transmitted at another interval, such as weekly or twice weekly, with a scaling up of the bandwidth of transmission. The high bandwidth transmission of the raw data can occur daily while the therapy device is being charged, such as at night during a regular charging period. In some embodiments, the raw data can be processed by the cloud and/or the physician into processed data and sent back to the therapy device.

In some embodiments, the system may optionally include a portable computing device 1006, such as a smart phone or tablet, to provide a secondary display and user interface for the patient and to run applications to more easily control the therapy device and view the raw and processed data. The portable computing device can be used to make patient or physician adjustments to the therapy device, such as adjusting the stimulation parameters and dosing, and can receive device state data from the therapy device, which includes data relating to the device, such as when the device was used, errors, therapy parameters such as amplitude and when they were set and delivered. In some embodiments, the portable computing device 1006 can receive processed data from the cloud I 002 through a cellular network and/or through an internet connection using WiFi, for example.

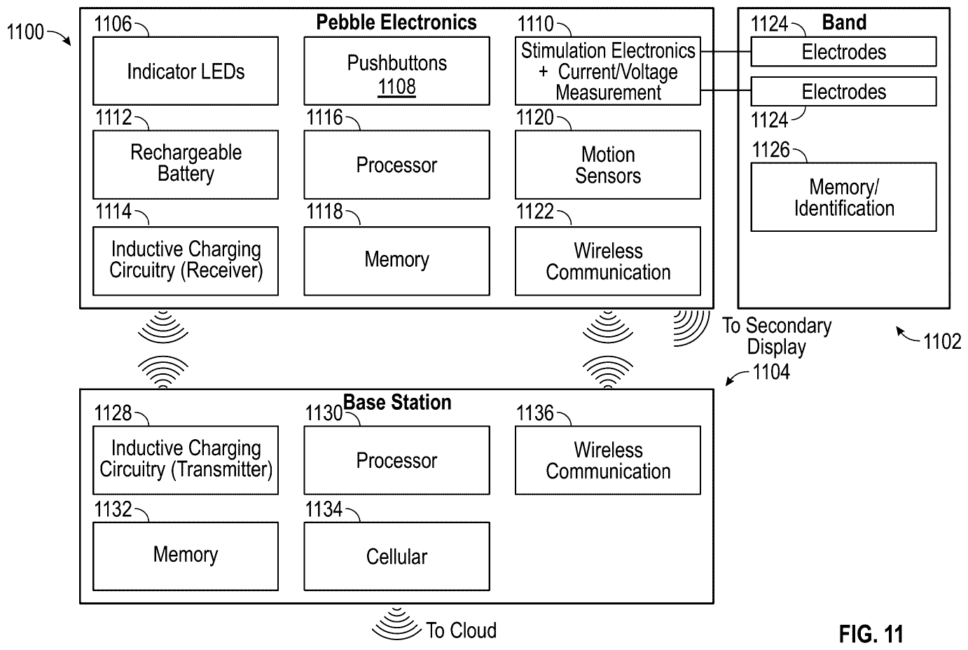

FIG. 11 illustrates the various components that can be included in a therapy unit 1100, band 1102, and base station 1104. These components are described in detail above and also below as one particular embodiment. For example, the therapy unit 1100 include one or more indicators 1106, which can be LEDs, and a user interface 1108, which can be push buttons, for example. The therapy unit 1100 can also have a stimulator 1110 with stimulation electronics and may include the capability to measure current and voltage. The therapy unit 1100 can also have a battery 1112, which may be rechargeable and can be recharged using charging circuitry 1114, which may be inductive. The therapy unit 1110 may further include a processor 1116 and memory 1118 to store and execute programs and instructions to accomplish the functions described herein. The therapy unit 1110 may also include sensors 1120, such as motion sensors, and a communications module 1122, which may be wireless and can communicate with the base station 1104 and/or a secondary display/computing device.

The band 1102 can have electrodes 1124 and may also include memory to store identification information or may include some other form of identifier 1126 as described herein.

The base station 1104 can include charging circuitry 1128, which may also be inductive and can transmit power to the complementary charging circuitry 1114 on the therapy unit 1100. The base station 1104 can also have a processor and memory for storing and executing instructions and programs. The base station 1104 can further include a communication module 1132, which may be cellular, to communicate with the cloud, and another communication module 1134, which may be wireless and used to communicate with the therapy unit.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−I% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “1 0” is disclosed, then “about I 0” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

What is claimed is:

1. A system for stimulating at least one nerve of a patient, the system comprising:
a band comprising:
a first surface configured to contact skin of the patient, the first surface comprising at least two skin electrodes,
a second surface,
at least two receiving contacts, and
an electrical circuit configured to be in electrical communication with the at least two skin electrodes and the at least two receiving contacts; and
a therapy unit comprising:
a stimulator configured to generate an electrical stimulation, the stimulator in electrical communication with the electrical circuit.
